(19) United States
(12) Patent Application Publication
(10) Pub. No.: US 2006/0058271 A1
(43) Pub. Date: Mar. 16, 2006

(54) Methods for screening, studying, and treating dissorders with a component of mercurial toxicity

(76) Inventors: Mark R. Geier, Silver Spring, MD (US); David A. Geier, Silver Spring, MD (US)

Correspondence Address: David A. Geier, 14 Redgate Court, Silver Spring, MD, 20905, US

(21) Appl. No.: 10/941,887

(22) Filed: Sep. 16, 2004

(51) Int. Cl. C07J 1/00; A61K 31/56

(52) U.S. Cl. 514/169; 552/502

(57) ABSTRACT

The present invention is related to a method, system or process for screening, studying and treating disorders which have a component of mercurial including: autism, autism spectrum disorders, ADD, ADHD, mental retardation, Asperger's syndrome, childhood psychoses, stammering, stuttering, tics, repetitive movements, eating disorders, sleep disorders, enuresis, disturbances of emotion, developmental language disorders, developmental speech disorders, developmental delay, Alzheimer's disease, diabetes, heart disease, obesity, ALS, nephritic syndrome, renal failure, asthma, systemic lupus, autoimmune thyroiditis, rheumatoid arthritis, arthritis, vasculitis, myelitis, glomerulonephritis, optic neuritis, infantile cerebral palsy, epilepsy, migraine, toxic encephalopathy, polyneuropathy, cerebral degenerations, anterior horn cell disease, spinocerebellar disease, extrapyramidal disease, and myopathy, and other related disorders. The invention of a method of screening, studying and treating of these disorders by utilizing the interaction of mercurial with the steroidgenesis pathway and its control by the hypothalamus-pituitary-adrenal axis is described.

### BACKGROUND

1. Field of the Invention

The present invention involves the fields of immunology and medicine, and more particularly relates to new investigative and treatment methods that relate to diseases in humans and other mammals that are caused by, worsened by, or otherwise affected by exposure to various mercurials.

2. Discussion of the Prior Art

Autism is a neurodevelopmental disorder characterized by impairments in social relatedness and communication, repetitive behaviors, abnormal movements, and sensory dysfunction. According to the most recent estimates published by the Centers for Disease Control and Prevention (CDC), it has been reported that approximately 1 in 150 children in the United States suffers from an autistic disorder, and far more males than females suffer from autistic disorders.^{1,2} Recent studies have reported that exposure to mercury can cause immune, sensory, neurological, motor, and behavioral dysfunctions similar to traits defining or associated with autism, and the similarities extend to neuroanatomy, neurotransmitters, and biochemistry.³⁻⁵

Thimerosal, a preservative added to many vaccines, has become a major source of mercury among children in the United States who, within their first two years, may have received a quantity of mercury that exceeded Federal Safety Guidelines.^{6,7} According to the CDC recommended immunization schedule in the United States during the 1990s, infants may have been exposed to 12.5 μg of ethylmercury at birth, 62.5 μg of ethylmercury at 2 months, 50 μg of ethylmercury at 4 months, 62.5 μg of ethylmercury at 6 months, and 50 μg of ethylmercury at 18 months, for a total of 237.5 μg of ethylmercury during the first 18 months of life, if all thimerosal-containing vaccines were administered.^{6,7}

Redwood et al.⁶ have estimated hair mercury concentrations expected to result from the recommended CDC childhood immunization schedule during the 1990s utilizing a one compartment pharmacokinetic model. The authors determined that modeled hair mercury concentrations in infants exposed to vaccinal thimerosal were in excess of the Environmental Protection Agency (EPA)'s safety guidelines of 1 part-per-million (ppm) for up to the first 365 days, with several peak concentrations within this period. The inventors have evaluated doses of mercury from thimerosal-containing childhood vaccines administered in accordance with the recommended CDC childhood immunization schedule during the 1990s in comparison the EPA and the Food and Drug Administration (FDA) safety guidelines for the oral ingestion of methylmercury, a similar compound to ethylmercury. We have reported that children received instantaneous doses of mercury from thimerosal-containing childhood vaccines that were many-fold in excess of the Federal Safety Guidelines.^{8,9}

Epidemiological studies conducted in the United States have examined the relationship between thimerosal-containing childhood vaccines and autistic disorders. It has been shown that children receiving thimerosal-containing childhood vaccines were 2- to 6-fold statistically significantly more likely to develop autistic disorders in comparison to children receiving thimerosal-free childhood vaccines.⁸⁻¹¹

Several recent studies have clinically evaluated the body-burden of heavy metals present in children with autistics disorders in comparison to normal children. Bradstreet et al.¹² have evaluated urinary heavy metals following three days of oral chelation with meso 2,3-dimercaptosuccinic acid (DMSA) in children with autistic disorders in comparison to a control population. It was determined that autistic children had statistically significantly approximately 6-fold higher urinary mercury concentrations than matched normal controls, whereas other heavy metals were present in similar urinary concentrations in both groups following three days of oral chelation with DMSA. In addition, in this study, urinary mercury concentrations were compared following three days of oral chelation with DMSA in matched vaccinated and unvaccinated normal children. It was observed that there were similar concentrations of urinary mercury in both groups following DMSA treatment. Holmes et al.¹³ have evaluated first baby haircuts from autistic children in comparison to controls. It was observed that the mercury levels in the first baby haircuts of children were inversely related to the severity of the autistic disorders of the children (i.e. the more severely affected the children are, the less mercury levels were present in their first baby haircuts). It has been hypothesized that these results are consistent with autistic children having biochemical differences than normal children, possibly as a result of genetic polymorphisms, resulting in children with autistic disorders having an increased body-burden of mercury in comparison to normal children.

Boris et al.¹⁴ have recently conducted genomic studies of children with autistic disorders in comparison to normal control populations. The authors have examined genes in pathways that are responsible for the synthesis of key biochemical molecules that are of functional relevance in the excretion and/or oxidative stress protection of mercury from the body. Specifically, the authors demonstrated that there was approximately a 2-fold statistically significant increase in homozygous genes of (MTHFR). This is of particular relevance because MTHFR is one of the key genes in the biochemical pathway involved with the synthesis of glutathione, a key molecule in the body's natural defenses against mercury, and those with homozygous genes for MTHFR have been found to have an enzyme that functions approximately 60% less than those with the standard MTHFR genes.

The understanding of the cause of the epidemic has allowed for the design of treatment modalities that address the mercury toxic component of these disorders. These therapies include methods to remove the mercury by such techniques as the use of chelating agents and by corrections in various biochemical pathways that lead to sulphydral-containing compounds that the body uses to rid itself of the mercury.¹⁵

Clarkson et al.¹⁶ have developed a mouse model to evaluate the neurotoxic effects of alkyl mercury exposure on different sexes. The authors reported that two-day-old mice were administered alkyl mercury at 4 mg of mercury/Kg/bodyweight (low dose), 8 mg of mercury/Kg bodyweight (high dose), or nor mercury. Animals were sacrificed 24 hours later, and matched sections of brain were prepared. The total number of mitotic figures in the external granule layer of the cerebellar cortex were recorded and classified as early (prophase and metaphase) or late (anaphase and telophase). Mercury concentrations in the brain for both males and females were 2.7 micrograms of mercury/gram at the high dose exposure and 1.8 micrograms of mercury at the low dose exposure. The authors determined that at the high dose, male and female mice had similarly reduced percentages of late mitotic figures compared with controls. At the lower dose, female mice were significantly much less affected in their percentages of late mitotic figures compared with male mice. The authors concluded males are considerably more sensitive to the neurotoxic effects of mercury, and that in some human fetal/infant population exposures to low dose alkyl mercury, it has been observed that males were more sensitive than females to psychomotor retardation.^{16,17} Muraoka and Itoh¹⁸ have investigated sex differences in the effects of mercury exposure on other organ systems. The authors reported that when doses of 0.3 to 2 mg/kg of mercuric chloride were intravenously administered to rats of the JCL-SD strain, acute renal tubular necrosis was produced in the straight portion of the proximal tubules with a pronounced sex difference, the male being more susceptible. Necrosis was inhibited by castration of male rats and promoted by testosterone pretreatment.

Researchers^{19,20} have investigated prenatal testosterone levels in children with autistic spectrum disorders. The authors examined 72 children with autism, including 23 children with Aspergers syndrome (i.e. these children have less serve autistic affects), 34 siblings, 88 fathers, 88 mothers, and sex and age-matched controls. The authors demonstrated that the more severely affected the children were the higher the levels of prenatal testosterone.

The precursors to testosterone and estrogen in the steroidgenic pathway are shown in FIG. 1. It is has been shown that one of the enzymes in the pathway to synthesizing testosterone, hydroxysteroid transferase (HST), which converts DHEA to DHEA-S, is known to have glutathione as a cofactor.²¹ Additionally, HST is known to be inhibited by mercury compounds (FIG. 2).¹¹ Studies have shown that glutathione levels tend to be lower in autistics and mercury levels are much higher.^{12,13,22}

The breakdown products of testosterone are shown in FIG. 3. Testosterone breakdown products are well known to play a major toxic role in male pattern baldness and in the development of benign prostatic hypertrophy.^{23,24} The FDA approved drug Finasteride, which blocks the breakdown of testosterone into 5-alpha-dihydrotestosterone, (DTH), has been shown to be highly effective in preventing and treating these conditions.^{23,24}

In addition to autistic disorders, a whole host of other diseases may also have a mercury toxic component such as Alzheimer's disease^{25,26}, diabetes²⁷, heart disease²⁸, obesity²⁹, ALS^{30,31}, asthma³², and various other forms of autoimmune disorders³³, all of which are very common in our mercury toxic population.³⁴

### SUMMARY

The present invention relates to the discovery that diseases in man and other mammals that are caused or influenced by exposure to mercurials also involve the steroidgenic pathway and of metabolites and breakdown products formed in the metabolism and breakdown pathways related to various molecules in the steriodogenesis pathway. Investigations of the interaction between the mercurials and this pathway allow new treatment methods for human and animal diseases. The methods of treatment described in this patent application provide for new ways to screen, study and treat disorders that have a component of mercurial toxicity by utilizing the interaction of mercurials with the steroidgenesis pathway and its control by the hypothalamus-pituitary-adrenal axis.

### BRIEF DESCRIPTION OF THE DRAWINGS

FIG. 1 is a description of the precursors to testosterone and estrogen in the steroidgenic pathway;

FIG. 2 shows the role of mercury and glutathione in the testosterone pathway;

FIG. 3 shows the breakdown pathway for testosterone;

FIG. 4 shows the regulation of the hypothalamus-pituitary-adrenal axis;

FIG. 5 shows the anatomy of the hypothalamus-pituitary-adrenal axis;

FIG. 6 describes all of the seemingly unrelated clinical treatments that have had some reported success in the treatment of autism, all of which can actually be seen to have one thing in common, i.e. they all in one way or another lower testosterone.

### DETAILED DESCRIPTION

It is clear that many that a significant number of persons suffering from mercury intoxication do not respond to the present treatment therapies that are designed to help eliminate mercury from the body. What is needed is another modality of clinical treatment to complement the work on eliminating mercury from these mercury toxic individuals. We purpose new ways to screen, study and treat disorders that have a component of mercurial toxicity by utilizing the interaction of mercurials with the steroidgenesis pathway and its control by the hypothalamus-pituitary-adrenal axis.

Experience from many areas of medicine has shown that, in diseases that can be attacked in multiple ways, the effects of such therapies are not only additive but are often strongly synergistic.

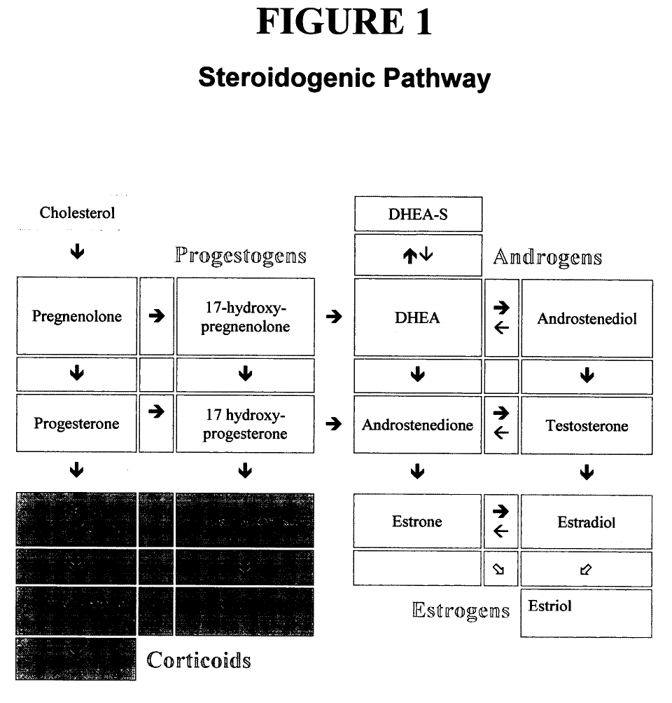

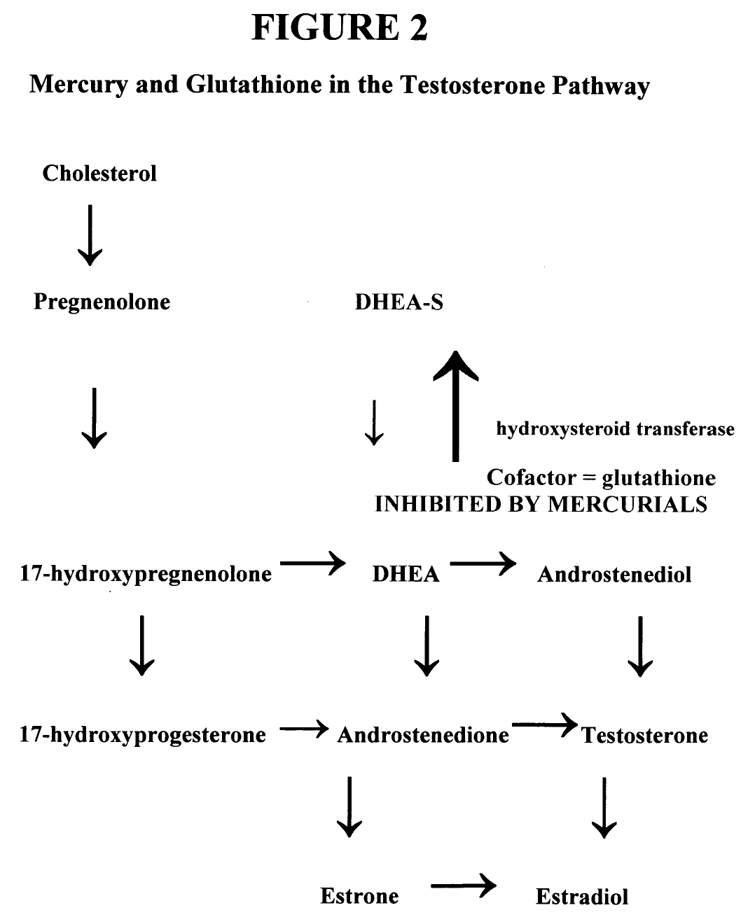

In FIG. 1 is a description of the precursors to testosterone and estrogen in the steroidgenic pathway. HST may well be significantly reduced or inhibited among those suffering from the chronic effects of mercury intoxication. Normally, most of the DHEA that is produced in the testosterone synthesis pathway is stored as DHEA-S thus reducing the amount that goes on to be made into androstrenediol and then into testosterone itself. Therefore, we purpose if HST were blocked by low glutathione and high mercury, then the pathway would be shifted to produce more testosterone and subsequently more testosterone breakdown products, and by administering glutathione and removing the mercury by chelation, it may be possible to improve the function of HST among those suffering from mercury intoxication.

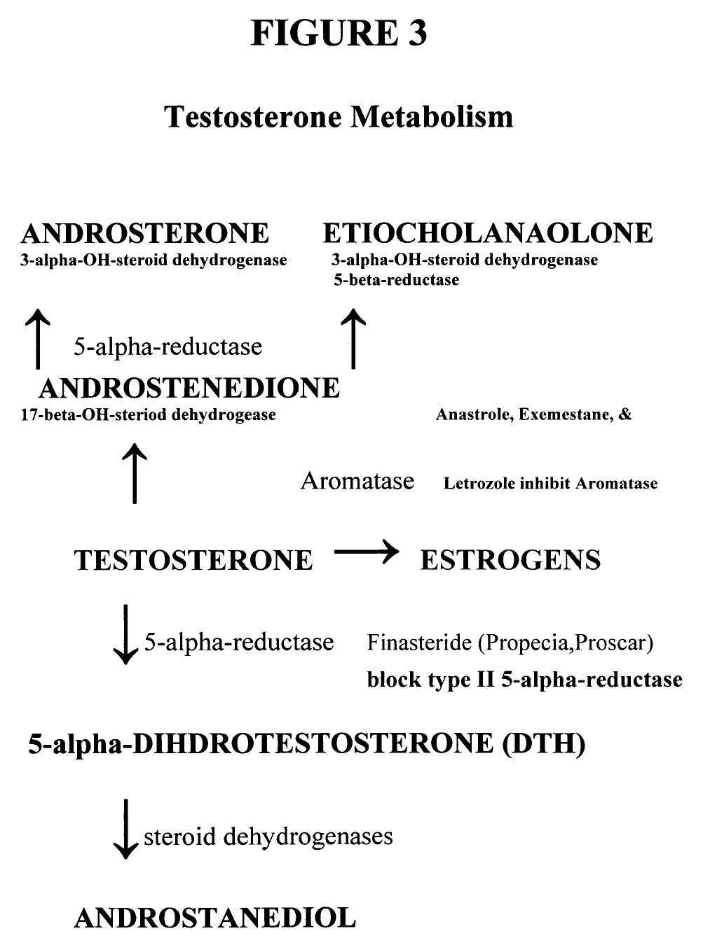

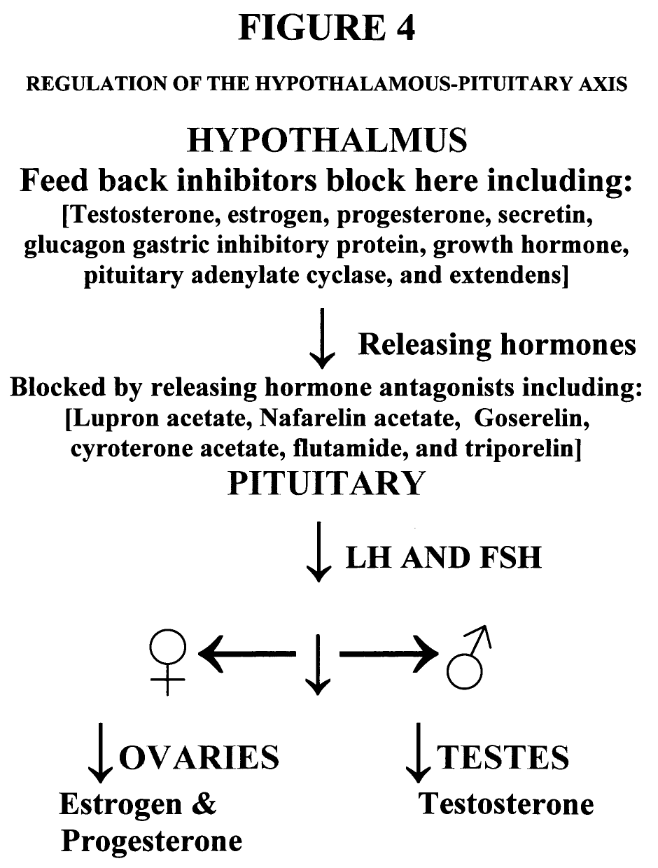

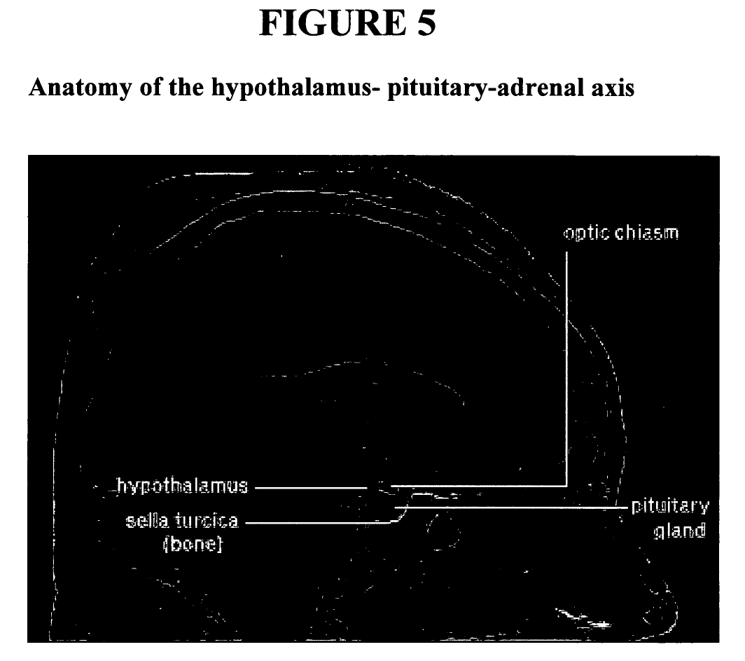

The breakdown products of testosterone and estrogen need to be tested to determine their effects on the neurotoxicity, immunotoxicity, genotoxicity, and general toxicity of mercury compounds. The breakdown pathway for testosterone is shown in FIG. 3. If these testosterone metabolic compounds are shown to potentate mercury toxcity, it is possible the same drugs might be effective in the treatment of mercury-associated disorders. Biochemical manipulations that favor the conversion of testosterone to estrogen also might well be shown in the tissue culture systems to protect cells from damage by mercurials. FDA approved anti-androgens such as Bicalutamide, Nolvadex, Nilandron and Flutamide might also protect neurons from damage from mercurials. Even the introduction or manipulation of the related corticosteroid pathways might be found to alter the toxicity of mercury compounds. Biochemical strategies, which are found to ameliorate the toxic effects of mercury compounds in tissue culture, could then be tried in animal models of mercury-associated toxicity prior to trials in humans.

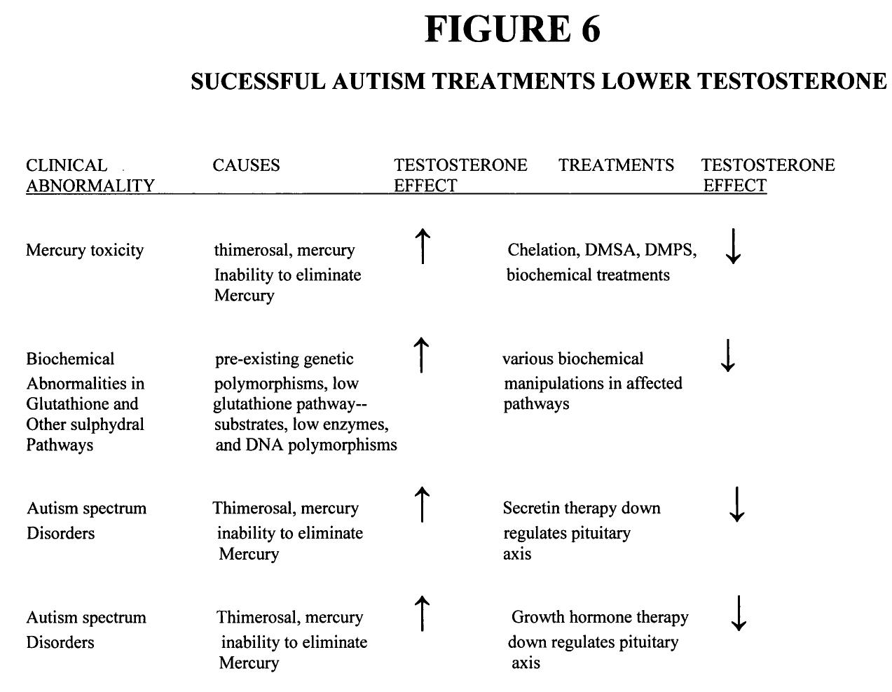

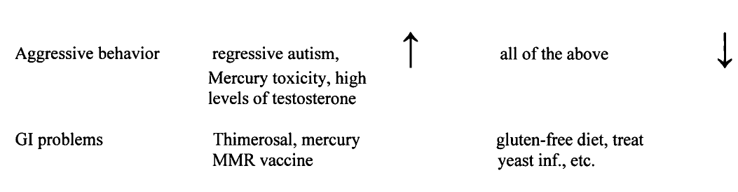

We believe that the potentating effect of testosterone and the protective effect of estrogen need to be studied in far more detail because essentially all of the seemingly unrelated clinical treatments that have had some reported success in the treatment of autism can actually be seen to have one thing in common, i.e. they all in one way or another lower testosterone, (FIG. 6). The kinetics of these effects needs to be determined. Additionally, the precursors to testosterone and estrogen in the steroidgenic pathway as shown (FIG. 1) need to be tested for their effects on the toxicity of mercury compounds on various cell lines in tissue culture, including but not limited to: neuronal cells, gonad cells, immune cells.

Another area for treatment for mercury intoxication may involve modulating the steroidogenic pathway through use of polypeptides of the vasoactive intestinal polypeptide (VIP)-secretin-glucogon family. All members of this polypeptide family possess a remarkable amino-acid sequence homology, and bind to G-protein-coupled receptors, whose signaling mechanism primarily involves AC/protein kinase A and phospholipase C/protein kinase C cascades, and these among others have been shown to inhibit the regulation of the hypothalamus-pituitary-adrenal axis. We purpose that drugs, including but not limited to, such as triptorelin, cyproterone, flutamide, Lupron acetate, Nafarelin acetate, and Goserelin, all of which are gonadotropin-releasing hormone agonists, may be of use in manipulating the steroidogenic pathway. They may be useful in the treatment of disorders resulting from mercury intoxication.

We also purpose that testing other enzymes in the steroid synthesis and metabolic pathways may be determine to be important among those suffering from mercury intoxication, if they are inhibited by mercurials or if they have sulphydral containing cofactors.

### REFERENCES

Citation of documents herein is not intended as an admission that any of the documents cited herein is pertinent prior art, or an admission that the cited documents is considered material to the patentability of any of the claims of the present application. All statements as to the date or representation as to the contents of these documents is based on the information available to the applicant and does not constitute any admission as to the correctness of the dates or contents of these documents.
1. Bertrand J, Mars A, Boyle C, Bove F, Yeargin-Allsopp M, Decoufle P. Prevalence of autism in a United States population: the Brick Township, N.J., investigation. Pediatrics 2001;108:1155-61.
2. Yeargin-Allsopp M, Rice C, Karapurkar T, Doernberg N, Boyle C, Murphy C. Prevalence of autism in a US metropolitan area. JAMA 2003;289:49-55.
3. Bernard S, Enayati A, Redwood L, Roger H, Binstock T. Autism: a novel form of mercury poisoning. Med Hypotheses 2001;56:462-71.
4. Bernard S, Enayati A, Roger H, Binstock T, Redwood L. The role of mercury in the pathogenesis of autism. Mol Psychiatry 2002;7 Suppl 2:S42-3.
5. Blaxill M F, Redwood L, Bernard S. Thimerosal and autism? A plausible hypothesis that should not be dismissed. Med Hypotheses 2004;62:788-94.
6. Redwood L, Bernard S, Brown D. Predicted mercury concentrations in hair from infant immunizations: cause for concern. Neurotoxicology 2001;22:691-7.
7. Ball L K, Ball R, Pratt R D. An assessment of thimerosal use in childhood vaccines. Pediatrics 2001;107:1147-54.
8. Geier M R, Geier D A. Thimerosal in childhood vaccines, neurodevelopment disorders, and heart disease in the United States. J Am Phys Surg 2003;8:6-11.
9. Geier D A, Geier M R. An assessment of the impact of thimerosal on neurodevelopmental disorders. Pediatr Rehabil 2003;6:97-102.
10. Geier M R, Geier D A. Neurodevelopmental disorders after thimerosal-containing vaccines: a brief communication. Exp Biol Med 2003;228:660-4.
11. Geier D A, Geier M R. A comparative evaluation of the effects of MMR immunization and mercury doses from thimerosal-containing childhood vaccines on the population prevalence of autism. Med Sci Monit 2004;10(3):PI33-9.
12. Bradstreet J, Geier D A, Kartzinel J J, Adams J B, Geier M R. A case-control study of mercury burden in children with autistic spectrum disorders. J Am Physicians Surg 2003;8:76-9.
13. Holmes A S, Blaxill M F, Haley B E. Reduced levels of mercury in first baby haircuts of autistic children. Int J Toxic 2003;22:277-85.
14. Boris M, et al. Association of 5,10-Methylenetetrathydrofolate reductase (MTHFR) gene polymorphisms with autistic spectrum disorders. J Am Phys Surg (in press).
15. Johnson S. Micronutrient accumulation and depletion in schizophrenia, epilepsy, autism and Parkinson's disease? Med Hypotheses 2001;56:641-5.
16. Clarkson T W, Nordberg G F, Sager P R. Reproductive and developmental toxicity of metals. Scand J Work Environ Health. 1985;11:145-54.
17. Grandjean P, Weihe P, White R F, Debes F. Cognitive performance of children prenatally exposed to “safe” levels of methylmercury. Environ Res 1998;77:165-72.
18. Muraoka Y, Itoh F. Sex difference of mercuric chloride-induced renal tubular necrosis in rats—from the aspect of sex differences in renal mercury concentration and sulfhydryl levels—. J Toxicol Sci 1980;5:203-14.
19. Manning J T, Baron-Cohen S, Wheelwright S, Sanders G. The 2nd to 4th digit ratio and autism. Dev Med Child Neurol 2001;43:160-4.
20. Lutchmaya S, Baron-Cohen S, Raggatt P, Knickmeyer R, Manning J T. 2nd to 4th digit ratios, fetal testosterone and estradiol. Early Hum Dev 2004;77:23-8.
21. Ryan R A, Carrol J. Studies on a 3beta-hydroxysteroid sulphotransferase from rat liver. Biochim Biophys Acta 1976;429:391-401.
22. James S J. Impaired transsulfuration and oxidative stress in autistic children: Improvement with targeted nutritional intervention. Fall DAN!™ 2003 Conference, Portland, Oregon, 3-5 Oct. 2003.
23. Ellis J A, Sinclair R, Harrap S B. Androgenetic alopecia: pathogenesis and potential for therapy. Expert Rev Mol Med 2002;2002:1-11.
24. Djavan B, Waldert M, Ghawidel C, Marberger M. Benign prostatic hyperplasia progression and its impact on treatment. Curr Opin Urol 2004;14:45-50.
25. Pendergrass J C, Haley B E, Vimy M J, Winfield S A, Lorscheider F L. Mercury vapor inhalation inhibits binding of GTP to tubulin in rat brain: similarity to a molecular lesion in Alzheimer diseased brain. Neurotoxicology 1997;18:315-24.
26. Pendergrass J C, Haley B E. Inhibition of brain tubulin-guanosine 5′-triphosphate interactions by mercury: similarity to observations in Alzheimer's diseased brain. Met Ions Biol Syst 1997;34:461-78.
27. Waly M, Olteanu H, Banerjee R, Choi S W, Mason J B, Parker B S, Sukumar S., Shim S, Sharma A, Benzecry J M, Power-Charnitsky V A, Deth R C. Activation of methionine synthase by insulin-like growth factor-1 and dopamine: a target for neurodevelopmental toxins and thimerosal. Mol Psychiatry 2004;9:358-70.
28. Guallar E, Sanz-Gallardo M I, van't Veer P, Bode P, Aro A, Gomez-Aracena J, Kark J D, Riemersma R A, Martin-Moreno J M, Kok F J; Heavy Metals and Myocardial Infarction Study Group. Mercury, fish oils, and the risk of myocardial infarction. N Engl J Med 2002;347:1747-54.
29. Kishida K, Kuriyama H, Funahashi T, Shimomura I, Kihara S, Ouchi N, Nishida M, Nishizawa H, Matsuda M, Takahashi M, Hotta K, Nakamura T, Yamashita S, Tochino Y, Matsuzawa Y. Aquaporin adipose, a putative glycerol channel in adipocytes. J Biol Chem. Jul. 7, 2000;275(27):20896-902.
30. Sillevis Smitt P A, de Jong J M. Animal models of amyotrophic lateral sclerosis and the spinal muscular atrophies. J Neurol Sci 1989;91:231-58.
31. Barber T E. Inorganic mercury intoxication reminiscent of amyotrophic lateral sclerosis. J Occup Med 1978;20:667-9.
32. Kazantzis G. The role of hypersensitivity and the immune response in influencing susceptibility to metal toxicity. Environ Health Perspect 1978;25:111-8.
33. Nakagawa R. Concentration of mercury in hair of diseased people in Japan. Chemosphere 1995;30:135-40.
34. Centers for Disease Control and Prevention. Blood and hair mercury levels in young children and women of childbearing age—United States, 1999. MMWR 2001;50:140-3.

What is claimed is:

1. Method of treating developmental disorders, including: autism, autism spectrum disorders, ADD, ADHD, mental retardation, Asperger's syndrome, childhood psychoses, stammering, stuttering, tics, repetitive movements, eating disorders, sleep disorders, enuresis, disturbances of emotion, developmental language disorders, developmental speech disorders, developmental delay, and other related disorders.
(I) Screening of the molecules in the steroidgenesis pathway and of metabolites and breakdown products formed in the metabolism and breakdown pathways related to various molecules in the steriodogenesis pathway for their ability to influence the ability of ethylmercury, methylmercury, inorganic mercury, and other mercurial compounds to damage, kill, metabolically affect or cause apoptosis in neurons, in fibroblasts, in immunological cells, in other cell types in tissue culture or in animal model systems. The screening will also be done in the estrogen pathway to determine which molecules are capable of protecting against the toxicity of various mercurials. The screening will determine which metabolites enhance or inhibit the effect of the mercurial compounds to affect neurons, fibroblasts, immune system cells and organs, and in other tissues and organs and at what concentrations they have their effects.
(II) Screening of the analogs, antagonist, metabolites, breakdown products, and other effector molecules in and related to the steriodogenesis pathway for their ability to influence the ability of ethyl-mercury, methyl-mercury, inorganic mercury, and other mercurial compounds to damage, kill, metabolically effect or cause apoptosis in neurons, in fibroblasts, in immunological cells, in other cell types in tissue culture or in animal model systems. This screening will determine which molecules enhance or inhibit the effect of the mercurial compounds to affect neurons, fibroblasts, immune system cells and organs, and in other tissues and organs and at what concentrations they have their effects.
(III) The use of the aforementioned molecules in the steroidgenesis pathway and of metabolites and breakdown products formed in the metabolism and breakdown pathways related to various molecules in the steriodogenesis pathway as treatments, both alone and in various combinations with themselves and in combination with treatments, methods, and therapies designed to remove the mercury by such techniques as the use of chelating agents and by corrections in various biochemical pathways that lead to sulfhlydral-containing compounds that the body uses to rid itself of the mercury.
(IV) The use of treatments, methods, and therapies designed to increase the level of glutathione, Fe2+, Co2+, Mn2+, or other cofactors for enzymes in the steroidgenesis and breakdown pathways related to various molecules in the steriodogenesis pathway, either alone or in combination with the use of the therapies, both individually and in various combinations as described in (III) above.
(V) The use of various molecules, agonists, antagonists and hormones to inhibit or effect the regulation of the hypothalamus-pituitary-adrenal axis. Examples of such molecules include but are not limited to secretin, and growth hormone which inhibit gonadotropin-releasing hormone, (GnRH). Additional the following drugs: triptorelin, cyproterone, and flutamide, Lupron acetate, Nafarelin acetate, Goserelin all of which inhibit gonadotropin-releasing hormone agonists, may be of use in manipulating the steroidogenic pathway and therefore their use for investigation and treatment, alone or in combination with each other or in combination with the treatments, methods, and therapies described in (I), (II), (III), and (iv) above are claimed.
(VI) Testosterone, and perhaps the binding sites for various other molecules in the steroidgenesis and degradation pathways contain sulphydral groups and thus have the potential to bind mercurials. Therefore the use for investigation and treatment of molecules, agonists, antagonists, hormones, and drugs that inhibit, modify or otherwise influence the binding of mercurials to these sites are claimed. This use is claimed for the use of these molecules alone, in combination with themselves and in combinations with the treatments, methods, and therapies described in (I), (II), (III), (IV), and (V) above are claimed.

2. Method of treating Alzheimer's disease, diabetes, heart disease, obesity, ALS, nephritic syndrome, renal failure, and asthma utilizing the methods described in item 1. above.

3. Method to treat various other forms of autoimmune disorders/hyper-immune disorders such as systemic lupus, autoimmune thyroiditis, rheumatoid arthritis, arthritis, vasculitis, myelitis, glomerulonephritis, and optic neuritis utilizing the methods described in item 1. above.

4. Method to treat other neurologic conditions such as infantile cerebral palsy, epilepsy, migraine, toxic encephalopathy, polyneuropathy, cerebral degenerations, anterior horn cell disease, spinocerebellar disease, extrapyramidal disease, and myopathy utilizing the methods described in item 1. above.

5. Methods described above to include, in addition to treating humans, the treatment in other mammals.

6. Method of treating gastrointestinal problems which occur alone or as are often are found in autism, autistic spectrum disorder, ADD, ADHD, mental retardation, Asperger's syndrome, childhood psychoses, and other related disorders. The methods of treatment of the gastrointestinal problems is as described previously in section#1. part I-VI of the what is claimed in this patent.

7. Method of treating asthma, asthma-like problems or other respiratory problems which occur alone or as are often are found in autism, autistic spectrum disorder, ADD, ADHD, mental retardation, Asperger's syndrome, childhood psychoses, and other related disorders. The methods of treatment of these respiratory problem is as described previously in section#1. part I-VI of the what is claimed in this patent.

8. Method of treating stroke and cardiovascular disease which involve testosterone and or mercury which may occur alone or as a part of other medical syndromes. The methods of treatment of these disorders is as described previously in section#1. part I-VI of the what is claimed in this patent.

9. Method of treating precocious puberty in males and females and other disorders resulting from high or early expressed testosterones, estrogens, FSH, LH, and other associated molecules and breakdown products of these molecules and associated molecules. The methods of treatment of these disorders is as described previously in section#1. part I-VI of the what is claimed in this patent.

10. The use of Lupron and its various deposition and non-deposition forms as describe in section #1 to 8 of what is claimed in this patent.

11. The use of Lupron in all of its forms as described in illustrative case report #1.

12. The use of the methods of treatment is as described previously in section#1. part I-VI of the what is claimed in this patent for not only treatment but also as diagnostic tools and as methods of monitoring of disease progress and treatment progress in all of the above described conditions.

13. We also claim everything named in claims #1-12, as described previously substituting cadmium for where ever mercury is discussed/mentioned.

14. We also claim everything named in claims #1-12, as described previously substituting arsenic for where ever mercury is discussed/mentioned.

15. We also claim everything named in claims #1-12, as described previously substituting lead for where ever mercury is discussed/mentioned.

16. We also claim everything named in claims #1-12, as described previously substituting antimony for where ever mercury is discussed/mentioned.

17. We also claim everything named in claims #1-12, as described previously substituting silver for where ever mercury is discussed/mentioned.

18. We also claim everything named in claims #1-12, as described previously substituting thallium for where ever mercury is discussed/mentioned.

19. We also claim everything named in claims #1-12, as described previously substituting tin for where ever mercury is discussed/mentioned.

20. We also claim everything named in claims #1-12, as described previously substituting aluminum for where ever mercury is discussed/mentioned.

21. We also claim everything named in claims #1-12, as described previously substituting magnesium for where ever mercury is discussed/mentioned.

22. We also claim everything named in claims #1-12, as described previously substituting manganese for where ever mercury is discussed/mentioned.

23. We also claim everything named in claims #1-12, as described previously substituting molybdenum for where ever mercury is discussed/mentioned.

24. We also claim everything named in claims #1-12, as described previously substituting copper for where ever mercury is discussed/mentioned.

25. We also claim everything named in claims #1-12, as described previously substituting nickel for where ever mercury is discussed/mentioned.

26. We also claim everything named in claims #1-12, as described previously substituting platinum for where ever mercury is discussed/mentioned.

27. We also claim everything named in claims #1-12, as described previously substituting thorium for where ever mercury is discussed/mentioned.

28. We also claim everything named in claims #1-12, as described previously substituting tungsten for where ever mercury is discussed/mentioned.

29. We also claim everything named in claims #1-12, as described previously substituting uranium for where ever mercury is discussed/mentioned.

30. We also claim everything named in claims #1-12, as described previously substituting all other heavy metals for where ever mercury is discussed/mentioned.

31. We also claim everything named in claims #1-12, as described previously substituting hypoxia for-where ever mercury is discussed/mentioned.

32. We also claim everything named in claims #1-12, as described previously substituting acidosis for where ever mercury is discussed/mentioned.

33. We also claim everything named in claims #1-12, as described previously substituting burns for where ever mercury is discussed/mentioned.

34. We also claim everything named in claims #1-12, as described previously substituting inducers of oxidative stress for where ever mercury is discussed/mentioned.

35. We also claim everything named in claims #1-12, as described previously substituting inducers of inflammation for where ever mercury is discussed/mentioned.

36. We also claim everything named in claims #1-12, as described previously substituting inducers of trauma for where ever mercury is discussed/mentioned.

37. We also claim everything named in claims #1-12, as described previously substituting inducers of hemorrhage for where ever mercury is discussed/mentioned.
